(19) United States
(12) Patent Application Publication
(10) Pub. No.: US 2023/0226170 A1
(43) Pub. Date: Jul. 20, 2023

(54) ENGINEERING CORONAVIRUS SPIKE PROTEINS AS VACCINE ANTIGENS, THEIR DESIGN AND USES

(71) Applicant: Greffex, Inc., Houston, TX (US)

(72) Inventors: Uwe D. STAERZ, Denver, CO (US); Daniel F. PRESTON, Denver, CO (US); Yan QI, Lone Tree, CO (US)

(21) Appl. No.: 17/998,607

(22) PCT Filed: May 12, 2021
(86) PCT No.: PCT/US2021/031974

§ 371 Date: Nov. 11, 2022

Related U.S. Application Data
(63) Continuation of application No. 63/023,599, filed on May 12, 2020.

(51) Int. Cl. A61K 39/215; C07K 14/005; A61P 31/14

(52) U.S. Cl. CPC A61K 39/215; C07K 14/005; A61P 31/14; C12N 2770/20022; C12N 2770/20034; C12N 2770/20071; A61K 2039/53

(57) ABSTRACT

A vaccine for preventing CoV infection includes at least one DNA, RNA or protein sequence for S protein with at least one modification which is a full deletion or partial deletion of the SI region or a partial or full replacement of the SI region. A method of vaccinating a mammal subject against infection from at least one group of CoV includes separating a broad group of CoV into homology groups, creating a modified S protein containing at least one modification at its S1 region, and identifying at least one consensus sequence for each homology group which has a sequence identity of greater than 60% to all other members of the homology group. The consensus sequence is a protein sequence for the modified S protein, a DNA sequence encoding the modified S protein, and an RNA sequence encoding the modified S protein.

### CROSS REFERENCE TO RELATED APPLICATIONS

This application claims priority to and is a non-provisional application of Provisional Application No. 63/023,599, filed on May 12, 2020, which is incorporated herein by reference in its entirety.

### FIELD

The disclosure relates to a vaccine platform for developing coronavirus vaccines, and more particularly to vaccines containing engineered coronavirus spike proteins. In another embodiment, the disclosure relates to methods for engineering coronavirus spike proteins and methods for making a vaccine containing an engineered coronavirus spike protein.

### SUBMISSION OF SEQUENCE LISTING

The contents of the electronic submission of the text file Sequence Listing which is named “Sequence Listing”, which was created May 11, 2021, and is 54 kb in size, is incorporated herein by reference in its entirety.

### BACKGROUND

Coronaviruses (CoVs) are classified into four genera: alpha-, beta-, gamma- and delta-coronaviruses. β-CoVs are enveloped, positive-strand RNA viruses capable of infecting mammals, generally bats and rodents, though many β-CoVs are known to infect humans as well. Infections with CoVs in humans and animals commonly produce mild to moderate upper-respiratory tract illnesses of short duration. Exceptions are the Severe Acute Respirator Syndrome (SARS-1), the Middle East Respiratory Syndrome (MERS) and the Wuhan-originating SARS-CoV-2 (SARS-2) (also referred to as COVID-19) that are characterized by severe and often lethal symptoms. The first cases of SARS-2 infections were seen in December 2019. As of Apr. 16, 2020, there were an estimated 632,000 cases reported and an estimated 31,000 deaths in the United States alone, as reported by the Center for Disease Control (CDC), resulting in a 4.9% lethality. SARS-2 is highly infectious to humans. The World Health Organization (WHO) declared the SARS-2 worldwide pandemic a Global Health Emergency on Jan. 30, 2020.

Specific treatments for SARS-2 are not available but under investigation. The best approach to prevent further spread of the disease is the development of specific vaccines. Herd immunity against SARS-2 is better achieved with immunization with a benign vaccine rather than by the natural infection with the active SARS-2 virus. One explanation for the low-level immune response seen in recuperating patients may be a function of exhaustive immune suppression by SARS-2. However, animal studies with traditional vaccines using an inactive version of the virus have suggested that inactivated virus vaccines might be especially prone to induction of antibody dependent enhancements (ADE) of the disease. For these vaccinations, Th2-type disease enhancement may be caused by anti-nucleocapsid (NP) response. It is desirable to develop a SARS-2 vaccine which does not stimulate ADE in vaccine recipients.

While social distancing has successfully suppressed the aggressive spread of SARS-2, it is anticipated that the reopening of societies will lead to a jump in infections in short order, as well as possible seasonal occurrences. Some regions have already seen jumps in infections with mutated versions of the SARS-2 virus. The overall mutation rates of SARS-related β-CoVs (SARSrs) have been calculated at as low as 0.1 mutations per generation. Despite the recent emergence of mutations, the SARS-2 virus seems to be similarly stable. It is desirable that any SARS-2 vaccine also provide protection against short-term variants.

Numerous animal as well as clinic trials with the related SARS- and MERS-CoVs have suggested that effective vaccines could be produced against more general β-CoV infections. SARS-2 (COVID-19) is the third lethal β-CoV that has jumped from animal hosts to humans. Considering that 1,800 SARSrs have already been identified in animals, some of which may eventually infect humans, it is desirable to also create group-specific SARSr vaccines to avert future pandemics.

There are further factors which support the need for a broadly reactive vaccine to provide protection against SARS-2 variants and SARSr viral strains. Analyzing SARSr animal sequences reveals that minor changes in the S protein receptor binding domain (RBD) sequence enhances the virus' binding to human ACE2, and such changes could therefore facilitate the jump of β-CoVs into the human population. The RBD sequence is located in a highly variable region of the S protein. Viruses are also known to mutate. With billions of people infected with SARS-2, numerous mutations are anticipated. A vaccine that induces immune responses against the more conserved regions of the S protein will provide broader protection against infection with different SARSrs.

### SUMMARY

In one embodiment, the disclosure provides a vaccine for preventing CoV infection. In accordance with embodiments of the present disclosure, a vaccine for preventing CoV infection comprises at least one sequence selected from the group consisting of a CoV DNA sequence which codes at least a portion of a modified S protein for the CoV, a CoV RNA sequence which codes at least a portion of a modified S protein for the CoV, and a protein sequence for at least a portion of a modified S protein of the CoV, wherein the modified S protein contains at least one modification.

In an embodiment, the S protein has an S1 region, and the at least one modification is selected from the group consisting of a full deletion of the S1 region, a partial deletion of the S1 region, a full replacement of the S1 region, and a partial replacement of the S1 region. In a further embodiment, the S1 region has at least one variable region, and the at least one modification is selected from the group consisting of a full deletion of the at least one variable region, a partial deletion of the at least one variable region, a full replacement of the at least one variable region, and a partial replacement of the at least one variable region. In still further embodiments, the CoV is a SARS-2 β-CoV and the at least one variable region is between amino acid residues 342 and 533. In another embodiment, the at least one variable region includes a receptor binding domain (RBD). In an embodiment, the at least one modification is a partial replacement of a variable region or a full replacement of the variable region, wherein the variable region is replaced by a peptide linker.

In an embodiment, the CoV is an a-CoV. In another embodiment, the CoV is a β-CoV. In a further embodiment, the sequence is a CoV DNA sequence, or a β-CoV DNA sequence. In another embodiment, the sequence is a CoV RNA sequence, or a β-CoV RNA sequence. In an embodiment, the RNA is mRNA. In still another embodiment, the sequence is a CoV protein sequence, or a β-CoV protein sequence. In a further embodiment, the sequence is a SARS-2 β-CoV sequence.

In one embodiment, the disclosure provides a modified CoV S protein. In accordance with embodiments of the present disclosure, a modified CoV S protein comprises an S1 region and at least one modification in the S1 region selected from the group consisting of a partial deletion of the S1 region, a full deletion of the S1 region, a partial replacement of the S1 region, and a full replacement of the S1 region.

In an embodiment, the S1 region has at least one variable region, and the at least one modification is selected from the group consisting of a full deletion of the at least one variable region, a partial deletion of the at least one variable region, a full replacement of the at least one variable region, and a partial replacement of the at least one variable region. In a further embodiment, the CoV is a SARS-2 β-CoV and the at least one variable region is between amino acid residues 342 and 533. In yet a further embodiment, the at least one variable region includes a receptor binding domain (RBD). In another embodiment, the at least one modification is a partial replacement of a variable region or a full replacement of the variable region, wherein the variable region is replaced by a peptide linker.

In one embodiment, the disclosure provides a method of making a vaccine to protect against infection by at least one CoV. In accordance with embodiments of the present disclosure, the method of making a vaccine to protect against infection by at least one CoV comprises creating a modified S protein comprising an S1 region and at least one modification in the S1 region, wherein the at least one modification is selected from the group consisting of a partial deletion of the S1 region, a full deletion of the S1 region, a partial replacement of the S1 region, and a full replacement of the S1 region.

In one embodiment, the disclosure provides a method of vaccinating a mammal subject against infection from at least one group of CoV. In accordance with embodiments of the present disclosure, the method of vaccinating a mammal subject against infection from at least one group of CoV comprises separating a broad group of CoV into homology groups based on similarities in the CoV S proteins; creating a modified S protein for each homology group, wherein the modified S protein comprises an S1 region and at least one modification selected from the group consisting of a partial deletion of the S1 region, a full deletion of the S1 region, a partial replacement of the S1 region, and a full replacement of the S1 region; and identifying at least one consensus sequence for each homology group which have a sequence identity in excess of 60% to all other members of the homology group, wherein the at least one consensus sequence is selected from the group consisting of a protein sequence for the modified S protein, a DNA sequence encoding the modified S protein, and an RNA sequence encoding the modified S protein.

In another embodiment, the method further includes preparing a viral vector including the at least one consensus sequence. In another embodiment, the step of separating a broad group of CoV into homology groups includes harvesting at least one CoV from an mammal. In another embodiment, the mammal is a human.

In a further embodiment, the S1 region has at least one variable region, and the at least one modification is selected from the group consisting of a full deletion of the at least one variable region, a partial deletion of the at least one variable region, a full replacement of the at least one variable region, and a partial replacement of the at least one variable region.

In an embodiment, the CoV is a SARS-2 β-CoV and the at least one variable region is between amino acid residues 342 and 533. In a further embodiment, the at least one variable region includes a receptor binding domain (RBD). In yet a further embodiment, the at least one modification is a partial replacement of a variable region or a full replacement of the variable region, wherein the variable region is replaced by a peptide linker.

In another embodiment, the CoV is an a-CoV. In a still further embodiment, the CoV is a β-CoV.

In an embodiment, the consensus sequence is a CoV DNA sequence, or a β-CoV DNA sequence. In yet another embodiment, the consensus sequence is a CoV RNA sequence, or a β-CoV RNA sequence. In an embodiment, the RNA is mRNA. In a further embodiment, the consensus sequence is a CoV protein sequence, or a β-CoV protein sequence. In an embodiment, the sequence is a SARS-2 β-CoV sequence.

In an embodiment, the method further comprises injecting the vaccine into the mammal subject.

### BRIEF DESCRIPTION OF THE DRAWINGS

FIG. 1 is a schematic showing functional portions of a SARS-2 β-CoV RNA segment which encodes the S protein, along with the portions of greatest variability and portions eliciting the greatest immune responses, in accordance with embodiments of the present disclosure.

FIG. 2 illustrates the designs of modified S proteins, in accordance with embodiments of the present disclosure.

FIG. 3 shows the activity of a MERS-CoV vaccine.

### DETAILED DESCRIPTION

Before any embodiments of the present disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is understood that the phraseology and terminology used herein is for the purpose of description and should not be regarded as limiting. The use of “including,” “comprising” or “having” and variations thereof herein is meant to encompass the items listed thereafter and equivalents thereof as well as additional items. The use of “including essentially” and “consisting essentially of” and variations thereof herein is meant to compass the items listed thereafter, as well as equivalents and additional items provided such equivalents and additional items to not essentially change the properties, use or manufacture of the whole. The use of “consisting of” and variations thereof herein is meant to include the items listed thereafter and only those items.

With reference to the drawings, like numbers refer to like elements throughout. It will be understood that, although the terms first, second, etc., may be used herein to describe various elements, components, regions, and/or sections, these elements, components, regions and/or sections should not be limited by these terms. These terms are used only to distinguish one element, component, region and/or section from another element, component, region and/or section. Thus, a first element, component, region or section could be termed a second element, component, region or section without departing from the disclosure.

The numerical ranges in this disclosure are approximate, and thus may include values outside of the range unless otherwise indicated. Numerical ranges include all values from and including the lower and the upper values (unless specifically stated otherwise), in increments of one unit, provided that there is a separation of at least two units between any lower value and any higher value. As an example, if a compositional, physical or other property, such as, for example, amount of a component by weight, etc., is from 10 to 100, it is intended that all individual values, such as 10, 11, 12, etc., and sub ranges, such as 10 to 44, 55 to 70, 97 to 100, etc., are expressly enumerated. For ranges containing explicit values (e.g., a range from 1, or 2, or 3 to 5, or 6, or 7), any subrange between any two explicit values is included (e.g., the range 1-7 above includes subranges 1 to 2; 2 to 6; 5 to 7; 3 to 7; 5 to 6, etc.). For ranges containing values which are less than one or containing fractional numbers greater than one (e.g., 1.1, 1.5, etc.), one unit is considered to be 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure.

Spatial terms, such as “beneath,” “below,” “lower,” “above,” “upper,” and the like, may be used herein for ease of description to describe one element's or feature's relationship to another element(s) or feature(s) as illustrated in the figures. It will be understood that the spatially relative terms are intended to encompass different orientations depending on the orientation in use or illustration. For example, if a device in the figures is turned over, elements described as “below” or “beneath” other elements or features would then be oriented “above” the other elements or features. Thus, the exemplary term “below” can encompass both an orientation of above and below. A device may be otherwise oriented (rotated 90° or at other orientations) and the spatially relative descriptors used herein interpreted accordingly.

As used herein, the term “and/or” includes any and all combinations of one or more of the associated listed items. For example, when used in a phrase such as “A and/or B,” the phrase “and/or” is intended to include both A and B; A or B; A (alone); and B (alone). Likewise, the term “and/or” as used in a phrase such as “A, B and/or C” is intended to encompass each of the following embodiments “A, B and C; A, B, or C; A or C; A or B; B or C; A and C; A and B; B and C; A (alone); B (alone); and C (alone).

In an embodiment, the present disclosure provides a method for preparing a vaccine for preventing at least one CoV infection, or at least one β-CoV infection, in a subject, particularly a mammal subject, and more specifically a human subject.

### Identifying β-CoVs

In accordance with embodiments of the present disclosure, a method for preparing a vaccine for preventing at least one β-CoV infection in a subject, particularly a mammal subject, and more specifically a human subject, comprises identifying at least one β-CoV from an animal host, particularly a mammal host. In a particularly embodiment, the method for preparing a vaccine for preventing at least one β-CoV infection in a subject, particularly a mammal subject, and more specifically a human subject, comprises identifying at least one β-CoV from a mammal host selected from the group consisting of a bat, a rat, a human, and combinations thereof. In an embodiment, the at least one β-CoV comprises at least one SARSr. In another embodiment, the at least one β-CoV comprises at least one SARS-2 β-CoV.

### Identifying Homology Groups

In accordance with embodiments of the present disclosure, a method for preparing a vaccine for preventing at least one β-CoV infection in a subject, particularly a mammal subject, and more specifically a human subject, comprises separating identified β-CoVs, such as those identified from an animal host, into homology groups based on similarities in genetic sequence and preparing at least one consensus sequence for each homology group. The homology groups can be based on similarities in the entirety of the β-CoVs' genetic sequences, multiple portions of the β-CoVs' genetic sequences, or a single portion of the β-CoVs' genetic sequences. The genetic sequences are selected from the group consisting of DNA sequences, RNA sequences, protein sequences, and combinations thereof. It will be understood that if a single β-CoV is identified, it is the sole member of a single homology group.

In a particular embodiment, the β-CoVs comprise a plurality of SARSrs, and the plurality of SARSrs are separated into 1, or at least 2, or at least 3, or at least 4, or at least 5 homology groups. In an embodiment, the homology groups are based on at least a portion, or at least two or more portions, or all, of the genetic sequence associated with the spike protein (S protein), the SARS receptor binding domain (RBD), an envelope protein, a nucleoprotein, and combinations thereof.

In a further embodiment, at least one SARS-2 β-CoV is identified and separated into at least one homology group.

In an embodiment, within each homology group, the genetic sequences have a sequence identity greater than or equal to 60%, or greater than or equal to 65%, or greater than or equal to 70%, or greater than or equal to 75%, or greater than or equal to 80%, or greater than or equal to 85%, or greater than or equal to 90%, or greater than or equal to 95%, or greater than or equal to 96%, or greater than or equal to 97%, or greater than or equal to 98%, or greater than or equal to 99% to all other members in the homology group.

In an embodiment, within each homology group, the genetic sequences have a sequence identity from greater than or equal to 60%, or greater than or equal to 65%, or greater than or equal to 70%, or greater than or equal to 75%, or greater than or equal to 80%, or greater than or equal to 85% to 90%, or 95%, or 96%, or 97%, or 98%, or 99%, or less than 100% to all other members in the homology group.

In an embodiment, the genetic sequences for each homology group define a distinct protein sequence for the homology group. In an embodiment, the distinct protein is selected from the group consisting of the S protein, an envelope protein, a nucleoprotein, and combinations thereof. In a further embodiment, the distinct protein is the S protein.

In a particular embodiment, a plurality of SARSrs are analyzed and separated into 5 homology groups, wherein, within each homology group, the genetic sequences have a sequence identify from greater than 65% to 99%.

An exemplary process for identifying homology groups and consensus sequences is now provided.

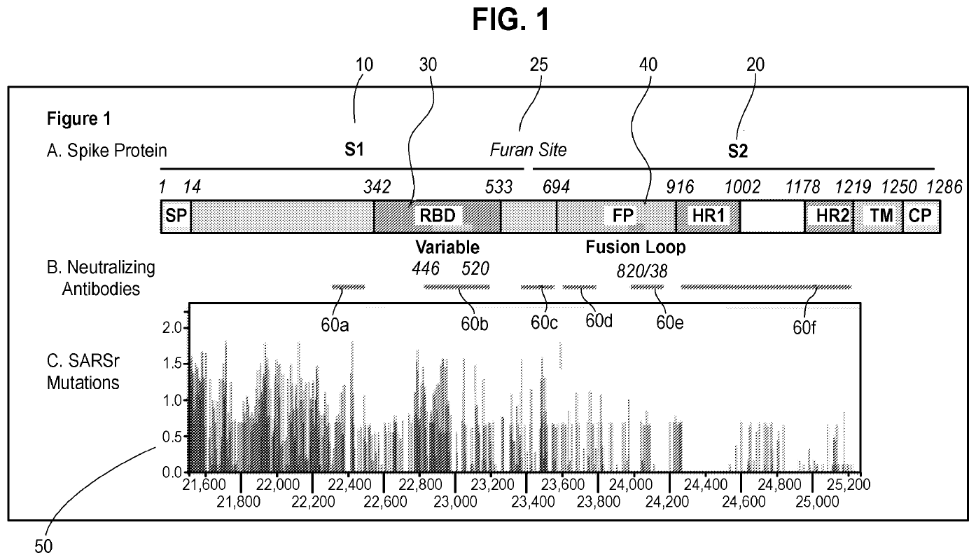

The SARS-2 β-CoV has a positive-sense, single-stranded RNA genome of about 30 kb and four structural proteins. One of the structural proteins is the spike (S) peplomer. These S proteins are found on the surface of the SARS-2 β-CoV and mediate cell receptor binding, and therefore determine the host tropism of the virus. The protein portion of the RNA which codes the S protein is divided into an S1 chain and an S2 chain, with the S1 chain 10 and the S2 chain 20 separated by a furan cut site 25, as shown in FIG. 1. The RBD 30 is located in the S1 chain 10. It was discovered that variations in the RBD influence the virus's binding to the angiotensin-converting enzyme 2 (ACE2), and an enhancement of this binding is through to facilitate the jump of the virus from animal hosts to human hosts. The membrane fusion section 40 is located in the S2 chain 20. Further shown in FIG. 1 are the heptapeptides HR1 and HR2, the transmembrane TM and the cytoplasmic domain of the S protein.

The SARS-2 β-CoV S protein is split into the S1 chain 10 and the S2 chain 20. Conformational changes in the S2 chain 20 lead to the fusion of the virus within the host cell. In combination with the S protein-encoding RNA sequence including the RBD, this makes the S protein-encoding RNA sequence a significant candidate for use in an anti-SARS-2 β-CoV vaccine regimen.

FIG. 1 also shows the S protein portions of the SARS-2 β-CoV which elicit greater immune responses (60). As shown, portion 60b overlaps with the RBD 30 and is a sizable portion, meaning there is significant immune response associated with the RBD 30. Portions 60d and 60e, while overlapping with the less-variable membrane fusion section 40, are smaller and therefore do not elicit as strong of an immune response. In analyzing the immune responses to different β-CoVs, such as SARS-CoV-1 and MERS-CoV, it was observed that antibodies that had the ability neutralize the activity of these coronaviruses could also bind to the more conserved areas of the S protein, namely within the S protein stem area within the S2 domain 20. As further illustrated in FIG. 1, aligning the S protein-encoding sequences of RNA from various SARSrs shows significant divergence throughout the gene (50). A vaccine based on present SARS-2 β-CoV RNA may therefore fail to efficiently protect against infections caused by other SARSrs. However, when the S protein-encoding sequences of RNA from a plurality of SARSrs are analyzed, the SARSrs can be separated into homology groups, as shown in Table 1.

| | | | | S Protein- | |
|---|---|---|---|---|---|
| | | | Closest to | Encoding | |
| | Number of | | Consensus* | RNA | RBD |
| Group | Sequences | Consensus | Accession # | Sequence | Sequence |
| Name | Analyzed | Sequence | (GenBank) | Identity | Identity |
| All | 4276 | | SARS-2 | 68% | 69% |
| Groups | | | (QJD07688.1) | | |
| SARS- | 1130 | SEQ. ID. | AAP13441.1 | 99% | 99% |
| CoV | | 1 | | | |
| WIV-1 | 56 | SEQ. ID. | AGZ48818.1 | 97% | 92% |
| | | 2 | | | |
| Bat | 19 | SEQ. ID. | ATO98169.1 | 94% | 98% |
| 2013 | | 3 | | | |
| YNLF | 71 | SEQ. ID. | AVP78031.1 | 82% | 95% |
| | | 4 | | | |
| SARS- | 3000 | SEQ. ID. | QJD07688.1 | 99% | 98% |
| CoV-2 | | 5 | | | |

*quantified sequence most closely resembling the consensus sequence of each group - generated using EMBOSS

To obtain the information in Table 1, sequences were found using ViPR and NCBI. Global alignment was done using Clustal Omega. Related alignments (>92%) were extracted to create the groupings, which were aligned using Clustal Omega and confirmed using BLAST multisequence alignments.

For the SARS-CoV group, 1130 sequences from GenBank and ViPR covering the original SARS-CoV-1 were analyzed. A couple of the sequences contained random inserts which are likely responsible for the gaps, but the small variants have all maintained antibody binding. For the SARS-CoV 2 group, greater than 3000 sequences were analyzed, including new clades 20H, 20I, and 20J (corresponding to the South African, California and UK variants, respectively). WIV-1 is a prominent SARSr in bats, but shown to replicate in human cells. 56 WIV-1 strains, including the RaTG13 strain thought to have given rise to SARS-2 β-CoV, were analyzed. Only 16 of the strains had complete CDS. Structures appeared steady between variants as shown by the NCBI Conserved Protein Domain Family cd21477 and Cn3D. For the YNLF group, 71 sequences (39 being complete CDS) where obtained from bats, pangolins and camels. These SARSr strains have less similarity to SARS-2 β-CoV than the WIV1 family, but have some strong similarities to the SARS-CoV group and SARS-CoV 2 group in certain regions. Global spike alignments are mediocre; however, RBD alignments show strong similarity. For the Bat2013 group, 19 samples with high similarity were analyzed. The Bat2013 group shows a higher variance than other groups, but many strains have shown cross-reactivity to the same antibodies.

### Consensus Sequence

In accordance with embodiments of the present disclosure, a method for preparing a vaccine for preventing at least one CoV, and preferably at least one β-CoV, infection in a subject, particularly a mammal subject, and more specifically a human subject, comprises identifying at least one consensus sequence for each homology group. A consensus sequence is a DNA, RNA or protein sequence developed for a group containing the statistically most frequent residue at each position in the sequence. In an embodiment, the consensus sequence for a homology group has at least 65%, or at least 70%, or at least 75%, or at least 80%, or at least 85%, or at least 90%, or at least 95%, or at least 99% commonality with each member of the corresponding homology group.

In a particular embodiment, a consensus sequence is a DNA sequence having a sequence identity greater than or equal to 60%, or greater than or equal to 65%, or greater than or equal to 70%, or greater than or equal to 75%, or greater than or equal to 80%, or greater than or equal to 85%, or greater than or equal to 90%, or greater than or equal to 95%, or greater than or equal to 96%, or greater than or equal to 97%, or greater than or equal to 98%, or greater than or equal to 99% to all other members in the corresponding homology group.

In a particular embodiment, a consensus sequence is an RNA sequence having a sequence identity greater than or equal to 60%, or greater than or equal to 65%, or greater than or equal to 70%, or greater than or equal to 75%, or greater than or equal to 80%, or greater than or equal to 85%, or greater than or equal to 90%, or greater than or equal to 95%, or greater than or equal to 96%, or greater than or equal to 97%, or greater than or equal to 98%, or greater than or equal to 99% to all other members in the corresponding homology group.

In a particular embodiment, a consensus sequence is a protein sequence having a sequence identity greater than or equal to 60%, or greater than or equal to 65%, or greater than or equal to 70%, or greater than or equal to 75%, or greater than or equal to 80%, or greater than or equal to 85%, or greater than or equal to 90%, or greater than or equal to 95%, or greater than or equal to 96%, or greater than or equal to 97%, or greater than or equal to 98%, or greater than or equal to 99% to all other members in the corresponding homology group.

In an embodiment, the consensus sequence for each homology group is selected from the group consisting of a DNA sequence, an RNA sequence, a protein sequence, and combinations thereof. In an embodiment, the consensus sequence for at least one of the homology groups is RNA. In a further embodiment, the RNA is mRNA.

In an embodiment, the β-CoVs analyzed are SARSrs. In a further embodiment, the SARSrs include at least one SARS-2 β-CoV separated into at least one homology group, and the consensus sequence of the at least one homology group is a DNA sequence, an RNA sequence, or a protein sequence. It will be appreciated that, in embodiments wherein a single SARSr, such as a single SARS-2 β-CoV, is identified, and the single SARSr is the only member of the homology group, a consensus sequence may be a DNA sequence, RNA sequence or protein sequence will have 100% commonality with the SARSr.

In an embodiment, the consensus sequence is a SARS-2 β-CoV DNA sequence, wherein the SARS-2 β-CoV DNA sequence is at least a portion of the S protein-encoding sequences.

In an embodiment, the consensus sequence is a SARS-2 β-CoV RNA sequence, wherein the SARS-2 β-CoV RNA sequence is at least a portion of the S protein-encoding sequence.

In an embodiment, the consensus sequence is a SARS-2 β-CoV protein sequence, wherein the SARS-2 β-CoV protein sequence is at least a portion of the S protein.

### Modification of Spike Protein

In an embodiment, the consensus sequence is a protein sequence for at least a portion of the S protein of a CoV or a CoV DNA or RNA sequence which encodes the at least a portion of the S protein, and the S protein contains at least one modification. That is, when the consensus sequence is a protein sequence, it is for the modified S protein. Similarly, when the consensus sequence is a DNA or RNA sequence, it encodes the modified S protein.

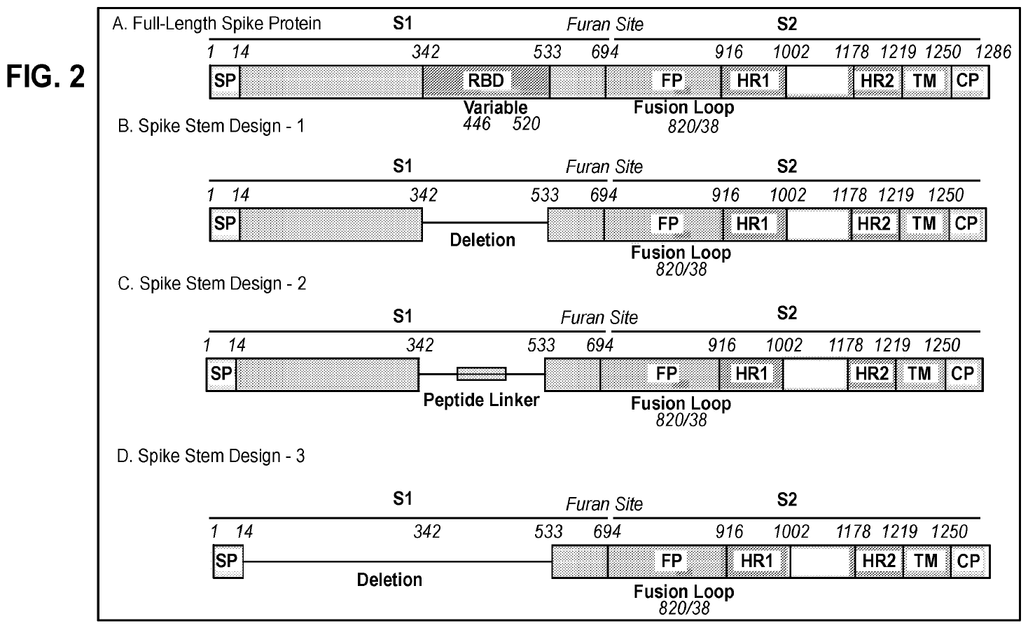

FIG. 2A is a schematic showing a full-length S protein sequence, while FIGS. 2B-2D are schematics showing exemplary modifications of the S protein sequence.

In an embodiment, the S1 region 10 of the S protein is defined as an area of the protein that starts at the amino-terminus of the protein end and ends at an amino acid sequence that corresponds to the peptidase recognition site 694 found in the SARS-2 S protein. In an embodiment, the at least one modification is in the S1 region 10 (i.e., the at least one modification is all, or at least a portion of, the protein sequence from the amino-terminus to the 694 amino acid sequence.

In an embodiment, the at least one modification is the deletion of the S1 region 10 in its entirety, e.g., from the amino-terminus end of the S protein to the 694 amino acid sequence. In another embodiment, the at least one modification is the deletion of one or more sequences, but less than the entirety, of the S1 region 10, as shown in FIGS. 2B and 2D.

In an embodiment, a “variable region” of the S protein is defined as an area of the S protein that shows high sequence variability when comparing SARSr sequences of naturally occurring SARS-CoV viruses and/or SARS-2 variants. Exemplary variable regions of the S protein are in FIG. 1, with the high frequency mutation regions shown in 50 being the exemplary variable regions. In an embodiment, the S protein has a single highly variable region referred to as “the variable region.”

In an embodiment, the at least one modification is the complete or partial deletion of at least one variable region, or the variable region, as shown in FIG. 2B.

In a particular embodiment, the variable region of the S protein is located between amino acid residues corresponding to the amino acid residues 342 and 533 of the SARS-2 (NC 045512.2). In such embodiment, the at least one modification is the complete or partial deletion of the variable region located between amino acid residues corresponding to the amino acid residues 342 and 533.

In a particular embodiment, the S protein has a variable region corresponding to most or all of the RDB region. In such an embodiment, the at least one modification is the complete or partial deletion of the RDB region. In one embodiment, the RDB region is located between amino acid residues corresponding to the amino acid residues at 342 and 533, or further between the amino acid residues at 446 and 520 of the SARS-2 (NC_045512.2).

In one embodiment, the at least one modification is the complete or total replacement of at least one variable region, or the variable region, wherein the variable regions are in accordance with any embodiment or combination of embodiments described above. The at least one variable region, or the variable region, may be replaced with a peptide linker sequence designed to bridge the gap created by the deletion, as shown in FIG. 2C.

### Viral Vector

In accordance with embodiments of the present disclosure, a method for preparing a vaccine for preventing at least one CoV infection, preferably a β-CoV infection, in a subject, particularly a mammal subject, and more specifically a human subject, comprises inserting the at least one consensus sequence into a viral vector. In an embodiment, the viral vector is an adenovirus vector component.

In order to minimize pre-existing and induced interfering anti-adenovirus immune responses, all endogenous genes have been deleted from the viral vector component, which is an adenovirus vector component. That is, in an embodiment, the viral vector component is a fully deleted (fd) adenovirus vector.

In an embodiment, the consensus sequence is a SARS-2 β-CoV DNA sequence which is at least a portion of the modified S protein-encoding sequences, a SARS-2 β-CoV RNA sequence which is at least a portion of the modified S protein-encoding sequences, or a SARS-2 β-CoV DNA sequence, wherein the SARS-2 β-CoV protein sequence is at least a portion of the modified S protein, wherein the modified S protein contains one or more modifications in accordance with any embodiment or combination of embodiments provided herein.

### Composition of the Vaccines

In an embodiment, the present disclosure provides a composition of a vaccine, and more particularly a vaccine to prevent against infection from CoVs, preferably β-CoVs, and more preferably SARSrs.

In accordance with embodiments of the present disclosure, the vaccine includes one or more consensus sequences derived from one or more CoVs, or β-CoVs, or preferably one or more SARSrs, carried on at least one viral vector. A consensus sequence may be in accordance with any embodiment or combination of embodiments described herein.

In an embodiment, the one or more consensus sequences is a β-CoV DNA sequence, RNA sequence, protein sequence, or combinations thereof, and preferably a SARSr DNA sequence, RNA sequence, protein sequence, or combinations thereof. Further, in an embodiment, the one or more consensus sequences is for a modified S protein.

In accordance with embodiments of the present disclosure, the one or more consensus sequences comprise at least one SARSr DNA or RNA sequence, or preferably at least one SARS-2 β-CoV DNA or RNA sequence. In an embodiment, the SARSr DNA or RNA sequence, or SARS-2 β-CoV DNA or RNA sequence, is at least a part of the modified S protein-encoding sequence.

The modified S protein is in accordance with any embodiment or combination of embodiments provided herein. In a particular embodiment, the S protein contains at least one modification which is the partial or complete deletion or replacement of at least one variable region, preferably at least one variable region in the S1 region. In a particular embodiment, one or more consensus sequences comprises at least one SARSr DNA or RNA sequences, and preferably at least one SARS-2 β-CoV DNA or RNA sequence, which is at least part of the modified S protein-encoding sequence, and the at least one modification is the partial or total removal or replacement of the RBD.

In an embodiment, expression of the consensus sequence is driven by a promotor. The promotor may be specific to the consensus sequence, animal being vaccinated, and the particular composition of the vaccine. In an embodiment, a promotor is selected from the group consisting of human cytomegalovirus immediate early promotor/enhancer, a poly-adenylation site derived from the human growth gene, the elongation factor 1-alpha, the phosphoglycerate kinase, ubiquitin C, beta actin genes, and combinations thereof. In embodiment, the promotor's activity may be influenced by a chemical, such as, but not limited to, an antibiotic. Tetracycline is a nonlimiting example of an antibiotic that influences a promotor's activity.

In a particular embodiment, the vaccine is specifically designed to prevent infection from at least SARS-2 β-CoV. In such an embodiment, the one or more consensus sequences includes at least one SARS-2 β-CoV DNA or RNA sequence. Preferably, the SARS-2 β-CoV DNA or RNA sequence is a modified S protein-encoding DNA or RNA sequence, wherein the modified S protein is an S protein containing at least one modification, and the at least one modification is at least one deletion or replacement in the S1 region. In a further embodiment, the SARS-2 β-CoV DNA or RNA sequence is an RNA sequence which is a modified S protein-encoding sequence (in part or in its entirety), wherein the S-protein has at least one modification which is a deletion or replacement of all or some of the RBD.

In an embodiment in which the consensus sequence is a SARS-2 β-CoV RNA sequence encoding the S protein (in part or in its entirety) in which at least part of a variable region of the S1 region is deleted or replaced, the SARS-2 β-CoV DNA sequence is human codon-optimized and expression of the specific RNA is driven by a human cytomegalovirus immediate early promotor/enhancer followed by a poly-adenylation site derived from the human growth gene. In other embodiments, the expression of the SARS-2 β-CoV RNA is driven by other promoters, such as, but not limited to, those derived from the elongation factor 1-alpha, the phosphoglycerate kinase, ubiquitin C, beta actin genes, and combinations thereof. In another embodiment, the expression of the SARS-2 β-CoV RNA is driven by a promoter whose activity can be influenced by a chemical, such as, but not limited to, the antibiotic tetracycline.

In further embodiments, the vaccine includes two or more consensus sequences in one or more viral vectors. In accordance with embodiments of the present disclosure, one consensus sequence is a SARS-2 β-CoV DNA, RNA or S protein sequence, and the vaccine includes at least one additional consensus sequence which is a SARSr DNA, RNA or protein sequence.

### Method of Vaccinating

In an embodiment the disclosure provides a method of vaccinating an animal subject, preferably a mammal subject, and more preferably a human subject against infection from at least one group of CoVs, preferably β-CoV.

In accordance with embodiments of the present disclosure, the method comprises modifying the S protein of a CoV, preferably a β-CoV, and more preferably a SARS-2 β-CoV, to create a modified S protein. The modified S protein includes at least one modification which is a full deletion, partial deletion, full replacement or partial replacement of the S1 region of the S protein. In a particular embodiment, the modified S protein includes at least one deletion or replacement of the S1 sequence from the amino-terminus to site 694. In a further embodiment, the modified S protein has a partial deletion of a variable region, a full deletion of a variable region, a partial replacement of a variable region, or a full replacement of a variable region. In an embodiment, the variable region is from amino residue 342 to 533 of the S protein. In a further embodiment, the variable region corresponds to some or all of the RBD. When the modification is a full or partial replacement of a variable region, the variable region may be replaced by a peptide linker.

The modified S protein may be used directly in a vaccine, such as, for example, alone or in combination with a viral vector. In further embodiments, a consensus sequence of CoV DNA or RNA which encodes the modified S protein may be used.

In accordance with embodiments of the present disclosure, the method comprises providing a vaccine comprising at least one viral vector comprising at least one CoV consensus sequence, or at least one β-CoV consensus sequence, or at least one SARSr consensus sequence, or at least one SARS-2 β-CoV consensus sequence, the consensus sequence being for a modified S protein. The at least one consensus sequence may be contained in at least one viral vector. The vaccine may further includes a plasmid. The viral vector (containing the consensus sequence) and plasmid are transfected into a packaging cell, and the packaging cell is encapsidated into a capsid.

In an embodiment, the at least one CoV consensus sequence is in accordance with any embodiment or combination or embodiments described herein. In a particular embodiment, the at least one CoV consensus sequence is a β-CoV DNA or RNA sequence which encodes at least a portion of the modified S protein.

The method further comprising injecting the consensus sequence into an animal subject, preferably a mammal subject, such as, for example, a human. In an embodiment, a single dose is sufficient to provide protection against at least one CoV, preferably at least one β-CoV, and more specifically provide protection against any CoVs having a sequence identity greater than or equal to 60%, or greater than or equal to 65%, or greater than or equal to 70%, or greater than or equal to 75%, or greater than or equal to 80%, or greater than or equal to 85%, or greater than or equal to 90%, or greater than or equal to 95%, or greater than or equal to 96%, or greater than or equal to 97%, or greater than or equal to 98%, or greater than or equal to 99% to at least one of the consensus sequence contained in the vaccine.

In other embodiments, two or more doses may be required to provide protection. In particular, two, or three, or four doses, is sufficient to provide protection against at least one CoV, and more particularly against any CoVs having a sequence identity greater than or equal to 60%, or greater than or equal to 65%, or greater than or equal to 70%, or greater than or equal to 75%, or greater than or equal to 80%, or greater than or equal to 85%, or greater than or equal to 90%, or greater than or equal to 95%, or greater than or equal to 96%, or greater than or equal to 97%, or greater than or equal to 98%, or greater than or equal to 99% to least one of the consensus sequences contained in the vaccine.

### EXAMPLES

### Example 1

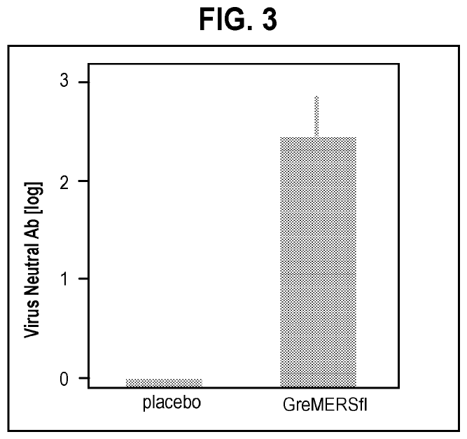

A control group of five mice (BALB/c mice) are vaccinated with a placebo. An experimental group of five mice (BALB/c mice) are vaccinated with 3×10⁷ genome equivalents of the GreMERSfl vaccine (containing a viral vector with a consensus sequence having at least 60% commonality with the EMX/2012 MERS-CoV) suspended in a vector suspension buffer (PBS, MgCl₂ 5 mM, EDTA 0.1 mM, sucrose 5%). The consensus sequence is the full-length S protein of the MERS-CoV. The control and experimental groups received a first intramuscular injection on day 1, followed by a booster dose on day 17 with the same respective preparations. On day 19, the two groups were intranasally infected with a LD50 of MERS. The groups were mixed on day 21. The mice were tested for the presence of antibodies neutralizing infection of test cells with the EMX/2012 MERS-CoV. As shown in FIG. 3, the control mice showed no antibodies, while the experimental group showed significant virus neutralization.

While multiple embodiments of a viral vector and associated vaccine have been described in detail herein, it should be apparent that modifications and variations thereto are possible, all of which fall within the true spirit and scope of the invention. In particular, while the present viral vectors and vaccines have been described in detail with respect to β-CoVs, and more particularly SARS-2 and SARSr viruses, it will be appreciated that the viral vectors and vaccines can be modified in accordance with the skill of one in the art to apply to other classes of coronaviruses, such as, for example, α-CoVs, γ-CoVs, and δ-CoVs. Further, since numerous modifications and changes will readily occur to those skilled in the art, it is not desired to limit the invention to the exact construction and operation shown and described, and accordingly, all suitable modifications and equivalents may be resorted to, falling within the scope of this disclosure.

1. A vaccine for preventing CoV infection, comprising:
at least one sequence selected from the group consisting of a CoV DNA sequence which codes at least a portion of a modified S protein for the CoV, a CoV RNA sequence which codes at least a portion of a modified S protein for the CoV, and a protein sequence for at least a portion of a modified S protein of the CoV, wherein the modified S protein contains at least one modification.

2. The vaccine of claim 1, wherein the S protein has an SI region, and the at least one modification is selected from the group consisting of a full deletion of the S1 region, a partial deletion of the S1 region, a full replacement of the S1 region, and a partial replacement of the S1 region.

3. The vaccine of claim 2, wherein the S1 region has at least one variable region, and the at least one modification is selected from the group consisting of a full deletion of the at least one variable region, a partial deletion of the at least one variable region, a full replacement of the at least one variable region, and a partial replacement of the at least one variable region.

4. The vaccine of claim 3, wherein the CoV is a SARS-2 β-CoV and the at least one variable region is between amino acid residues 342 and 533.

5. The vaccine of claim 4, wherein the at least one variable region includes a receptor binding domain (RBD).

6. The vaccine of claim 1, wherein the at least one modification is a partial replacement of a variable region or a full replacement of the variable region, wherein the variable region is replaced by a peptide linker.

7. The vaccine of claim 1, wherein the CoV is an α-CoV.

8. The vaccine of claim 1, wherein the CoV is a β-CoV,

9. The vaccine of claim 1, wherein the sequence is a CoV DNA sequence.

10. The vaccine of claim 9, wherein the CoV DNA sequence is a β-CoV DNA sequence.

11. The vaccine of claim 1, wherein the sequence is a CoV RNA sequence.

12. The vaccine of claim 11, wherein the CoV RNA sequence is a 13-CoV RNA sequence.

13. The vaccine of claim 11, wherein the RNA is mRNA

14. The vaccine of claim 1, wherein the sequence is a CoV protein sequence.

15. The vaccine of claim 14, wherein the sequence is a β-CoV protein sequence.

16. The vaccine of claim 1, wherein the sequence is a SARS-2 β-CoV sequence.

17. A modified CoV S protein comprising an S1 region and at least one modification in the S1 region selected from the group consisting of a partial deletion of the S1 region, a full deletion of the S1 region, a partial replacement of the S1 region, and a full replacement of the S1 region.

18. The modified CoV S protein of claim 17, wherein the S1 region has at least one variable region, and the at least one modification is selected from the group consisting of a full deletion of the at least one variable region, a partial deletion of the at least one variable region, a full replacement of the at least one variable region, and a partial replacement of the at least one variable region.

19. The modified CoV S protein of claim 18, wherein the CoV is a SARS-2 β-CoV and the at least one variable region is between amino acid residues 342 and 533.

20. The modified CoV S protein of claim 19, wherein the at least one variable region includes a receptor binding domain (RBD).

21. The modified CoV S protein of claim 18, wherein the at least one modification is a partial replacement of a variable region or a full replacement of the variable region, wherein the variable region is replaced by a peptide linker.

22. A method of making a vaccine to protect against infection by at least one CoV, the method comprising:
creating a modified S protein comprising an S1 region and at least one modification in the S1 region, wherein the at least one modification is selected from the group consisting of a partial deletion of the S1 region, a full deletion of the S1 region, a partial replacement of the S1 region, and a full replacement of the S1 region.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)


SEQUENCE LISTING (5 sequences)

SEQ ID NO: 1 - protein, 1255 aa, SARS-CoV
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 2 - protein, 1256 aa, SARSr
MKLLVLVFATLVSSYTIEKCLDFDDRTPPANTQFLSSHRGVYYPDDIFRSNVLHLVQDHFLPFDSNVTRFITFGLNFDNPIIPFKDGIYFAATEKSNVIRGWVFGSTMNNKSQSVIIMNNSTNLVIRACNFELCDNPFFVVLKSNNTQIPSYIFNNAFNCTFEYVSKDFNLDLGEKPGNFKDLREFVFRNKDGFLHVYSGYQPISAASGLPTGFNALKPIFKLPLGINITNFRTLLTAFPPRPDYWGTSAAAYFVGYLKPTTFMLKYDENGTITDAVDCSQNPLAELKCSVKSFEIDKGIYQTSNFRVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFTGCVLAWNTRNIDATQTGNYNYKYRSLRHGKLRPFERDISNVPFSPDGKPCTPPAFNCYWPLNDYGFYITNGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNTSSEVAVLYQDVNCTDVPVAIHADQLTPSWRVYSTGNNVFQTQAGCLIGAEHVDTSYECDIPIGAGICASYHTVSSLRSTSQKSIVAYTMSLGADSSIAYSNNTIAIPTNFSISITTEVMPVSMAKTSVDCNMYICGDSTECANLLLQYGSFCTQLNRALSGIAVEQDRNTREVFAQVKQMYKTPTLKDFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGSCDVVIGIINNTVYDPLQPELDSFKEELDKYFKNHTSPDVDLGDISGINASVVNIQKEINRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 3 - protein, 1241 aa, SARS
MKILIFAFLVTLVKAQEGCGLISRKPQPKMAQVSSSRRGVYYNDDIFRSDVLHLTQDYFLPFDSNLTQYFSLNVDSDRYTYFDNPILDFGDGVYFAATEKSNVIRGWIFGSTFDNTTQSAVIVNNSTHIIIRVCNFNLCKEPMYTVSRGTQQSSWVYQSAFNCTYDRVERSFQLDTAPKTGNFKDLREYVFKNRDGFLSVYQTYTAVNLPRGLPTGFSVLRPILKLPFGINITSYRVVMAMFSQTTSNFLPESAAYYVGNLKYTTFMLSFNENGTITDAIDCAQNPLAELKCTIKNFNVSKGIYQTSNFRVSPTQEVIRFPNITNRCPFDKVFNASRFPNVYAWERTKISDCVADYTVLYNSTSFSTFKCYGVSPSKLIDLCFTSVYADTFLIRSSEVRQVAPGETGVIADYNYKLPDDFTGCVIAWNTAKQDQGQYYYRSSRKTKLKPFERDLSSDENGVRTLSTYDFYPTVPIEYQATRVVVLSFELLNAPATVCGPKLSTGLVKNQCVNFNFNGLKGTGVLTDSSKRFQSFQQFGRDMSDFTDSVRDPQTLQILDITPCSFGGVSVITPGTNASSEVAVLYQDVNCTDVPTAIRADQLTPAWRVYSTGINVFQTQAGCLIGAEHVNASYECDIPIGAGICASYHTASTLRSVGQKSIVAYTMSLGAENSIAYANNSIAIPTNFSISVTTEVMPVSMSKTSVDCTMYICGDSQECSNLLLQYGSFCTQLNRALTGIAIEQDKNTQEVFAQVKQMYKTPAIKDFGGFNFSQILPDPSKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGRGYHLMSFPQAAPHGVVFLHVTYVPSQEKNFTTAPAICHEGKAYFPREGVFVSNGTSWFITQRNFYSPQIITTDNTFVAGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMATILLCCMTSCCSCLKGACSCGSCCKFDEDDSEPVLKGVKLHYT

SEQ ID NO: 4 - protein, 1246 aa, SARS
MLFFLFLQFALVNSQCVNLTGRTPLNPNYTNSSQRGVYYPDTIYRSDTLVLSQGYFLPFYSNVSWYYSLTTNNAATKRTDNPILDFKDGIYFAATEHSNIIRGWIFGTTLDNTSQSLLIVNNATNVIIKVCNFDFCYDPYLSGYYHNNKTWSIREFAVYSSYANCTFEYVSKSFMLNISGNGGLFNTLREFVFRNVDGHFKIYSKFTPVNLNRGLPTGLSVLQPLVELPVSINITKFRTLLTIHRGDPMPNNGWTAFSAAYFVGYLKPRTFMLKYNENGTITDAVDCALDPLSETKCTLKSLTVQKGIYQTSNFRVQPTQSVVRFPNITNVCPFHKVFNATRFPSVYAWERTKISDCIADYTVFYNSTSFSTFKCYGVSPSKLIDLCFTSVYADTFLIRFSEVRQVAPGQTGVIADYNYKLPDDFTGCVIAWNTAKQDVGNYFYRSHRSTKLKPFERDLSSDENGVRTLSTYDFNPNVPLEYQATRVVVLSFELLNAPATVCGPKLSTQLVKNQCVNFNFNGLKGTGVLTDSSKRFQSFQQFGKDASDFIDSVRDPQTLEILDITPCSFGGVSVITPGTNTSLEVAVLYQDVNCTDVPTTIHADQLTPAWRIYATGTNVFQTQAGCLIGAEHVNASYECDIPIGAGICASYHTASILRSTSQKAIVAYTMSLGAENSIAYANNSIAIPTNFSISVTTEVMPVSMAKTSVDCTMYICGDSIECSNLLLQYGSFCTQLNRALSGIAIEQDKNTQEVFAQVKQIYKTPPIKDFGGFNFSQILPDPSKPSKRSFIEDLLFNKVTLADAGFIKQYGDCLGGISARDLICAQKFNGLTVLPPLLTDEMIAAYTAALISGTATAGWTFGAGAALQIPFAMQMAYRFNGIGVTQNVLYENQKLIANQFNSAIGKIQESLTSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYIPSQEKNFTTAPAICHEGKAHFPREGVFVSNGTHWFVTQRNFYEPKIITTDNTFVSGNCDVVIGIINNTVYDPLQPELDSFKEELDKYFKNHTSPDIDLGDISGINASVVNIQKEIDRLNEVARNLNESLIDLQELGKYEQYIKWPWYVWLGFIAGLIAIVMVTILLCCMTSCCSCLKGCCSCGSCCKFDEDDSEPVLKGVKLHYT

SEQ ID NO: 5 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT